(19) United States
(12) Patent Application Publication
(10) Pub. No.: US 2020/0393698 A1
(43) Pub. Date: Dec. 17, 2020

(54) CONTACT LENS SOLUTION FOR ENHANCING MOIST SENSATION AND CONTACT LENS PRODUCT

(71) Applicant: PEGAVISION CORPORATION, TAOYUAN CITY (TW)

(72) Inventor: WEI-AN YEH, Taoyuan City (TW)

(21) Appl. No.: 16/871,158

(22) Filed: May 11, 2020

(30) Foreign Application Priority Data
Jun. 17, 2019 (TW) 108120918

(51) Int. Cl. G02C 7/04; G02B 1/04; C11D 3/00

(52) U.S. Cl. CPC G02C 7/04; C11D 3/0078; G02B 1/043

(57) ABSTRACT

A contact lens solution for enhancing moist sensation and a contact lens product are provided. The contact lens solution includes a comfort enhancing ingredient. The content of the comfort enhancing ingredient is between 0.01 wt % and 3 wt % based on 100 wt % of the contact lens solution. The comfort enhancing ingredient has one or more glutamic acid monomers, and has a number average molecular weight between 3,000 Daltons and 1,500,000 Daltons to allow the contact lens solution to have a viscosity between 1 cPs and 3 cPs.

### CROSS-REFERENCE TO RELATED PATENT APPLICATION

This application claims the benefit of priority to Taiwan Patent Application No. 108120918, filed on Jun. 17, 2019. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a contact lens solution, and more particularly to a contact lens solution for enhancing moist sensation, such as a package solution, a storage solution, a cleaning solution or a care solution, and a contact lens product using the same.

### BACKGROUND OF THE DISCLOSURE

Consumer electronic products such as smartphones and computers are frequently used in today's information society, resulting in an increase in the myopia population and a decrease in the average age of the myopia population. In consideration of user convenience and aesthetics, it is generally a good choice for people with myopia to wear contact lenses.

Since contact lenses are mostly worn by people with myopia for a long period of time, it is very important to improve the wearing comfort of contact lenses. The wearing comfort of contact lenses is associated with oxygen permeability, moisture retention, and lubricity of the contact lenses. Although the material of the contact lenses have been constantly improved to enhance oxygen permeability, moisture retention, and lubricity, in different use environments or with different eye conditions, the eyes of a user may still suffer from corneal injury or lesions caused by corneal hypoxia and dehydration with an increase of wear time, especially in an air-conditioned room for a long period of time.

Since contact lens solutions provided with glycerol merely reduces or relieves the discomfort, there is a need for a novel ophthalmic composition used on contact lenses to increase the wearing comfort of the contact lens and maintain the cornea in a healthy state.

### SUMMARY OF THE DISCLOSURE

In response to the above-referenced technical inadequacies, the present disclosure provides a contact lens solution for enhancing moist sensation, which can result in a comfortable sensation in eyes for a long period of time when wearing contact lenses, and a contact lens product using the contact lens solution.

In one aspect, the present disclosure provides a contact lens solution for enhancing moist sensation which includes a comfort enhancing ingredient. The content of the comfort enhancing ingredient is between 0.01 wt % and 3 wt % based on 100 wt % of the contact lens solution. The comfort enhancing ingredient has one or more glutamic acid monomers, and has a number average molecular weight between 3,000 Daltons and 1,500,000 Daltons. The contact lens solution has a viscosity between 1 cPs and 3 cPs.

In certain embodiments, the comfort enhancing ingredient is at least one selected from gamma-polyglutamic acid (γ-PGA) and its salts and gamma-polyglutamate.

In certain embodiments, the contact lens solution further includes a surfactant. The content of the surfactant is between 0.01 wt % and 2 wt % based on 100 wt % of the contact lens solution. The surfactant is at least one selected from polysorbate, an alkyl sulfosuccinate, polyethylene glycol, polyoxypropylene glycol and sodium dodecyl sulfate.

In certain embodiments, the contact lens solution further includes a buffering agent. The content of the buffering agent is between 0.01 wt % and 5 wt % based on 100 wt % of the contact lens solution. The buffering agent is a borate buffer or a phosphate buffer.

In certain embodiments, the borate buffer includes at least one of boric acid, sodium chloride and sodium borate.

In certain embodiments, the borate buffer includes at least one of sodium chloride, sodium dihydrogen phosphate, disodium hydrogen phosphate, potassium dihydrogen phosphate and dipotassium hydrogen phosphate.

In certain embodiments, the contact lens solution further includes a hydrophilic polymer. The content of the hydrophilic polymer is between 0.01 wt % and 5 wt % based on 100 wt % of the contact lens solution. The hydrophilic polymer is at least one selected from polyvinyl alcohol, cellulose and its derivatives, polyethylene glycol, palmitic acid, polyvinylpyrrolidone and its copolymers, 2-methacryloyloxyethyl phosphorylcholine and its copolymers, and hyaluronic acid.

In certain embodiments, the contact lens solution further includes a functional additive. The content of the functional additive is between 0.01 wt % and 5 wt % based on 100 wt % of the contact lens solution. The functional additive is an antimicrobial agent, a vitamin or the combination thereof.

In certain embodiments, the contact lens solution has a pH of 6 to 8 and an osmolality of 250 Osmol/Kg to 450 Osmol/Kg.

In another aspect, the present disclosure provides a contact lens product which includes a package provided with the contact lens solution as mentioned above and a contact lens. The contact lens is immersed in the contact lens solution and is sealed in the package.

One of the advantages of the present disclosure is that the contact lens solution for enhancing moist sensation and the contact lens product, in which the comfort enhancing ingredient is present in an amount of 0.01-3 wt % and has one or more glutamic acid monomers, can increase the wearing comfort of contact lenses as well as maintain healthy corneas.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 is a perspective view of a contact lens product of the present disclosure.

FIG. 2 is a cross-sectional view of the contact lens product of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

Unless defined otherwise, all technical and scientific terms used herein have the same meanings as commonly understood by one of ordinary skill in the art to which this invention belongs. As used herein, the singular forms “a,” “an,” and “the,” are intended to include the plural forms.

Unless indicated otherwise, all percentages disclosed herein are in weight percent. The upper and lower limits of these smaller ranges may independently be included in the smaller ranges and are also encompassed within the disclosure, subject to any specifically excluded limit in the stated range.

In order to increase the wearing comfort of contact lenses, the present disclosure provides a contact lens solution that mainly includes an ophthalmically acceptable liquid medium (e.g., water) and a comfort enhancing ingredient. The content of the liquid medium can be between 75 wt % and 99 wt %, preferably between 85 wt % and 99 wt %, based on 100 wt % of the contact lens solution. The contact lens solution of the present disclosure can serve as a package solution, a storage solution, a cleaning solution or a care solution, but it is not limited thereto.

In use, a contact lens can be in contact with the contact lens solution in a suitable manner, so as to allow an effective amount of the comfort enhancing ingredient to adhere onto the contact lens, and then be worn onto a human eye. For example, the contact lens can be immersed in the contact lens solution for a period of time, or the contact lens solution can be applied in drops to a surface of the contact lens. However, the present disclosure is not limited to the above examples. As used herein, the term “effective amount” is an amount that can deliver a sufficient amount of the comfort enhancing ingredient to the contact lens to produce moisture retention and lubrication effects.

In the present embodiment, the comfort enhancing ingredient at least has one or more glutamic acid monomers, and has high water absorption property and high viscosity. The contact lens solution has a viscosity between 1 cPs and 3 cPs in the presence of the comfort enhancing ingredient. More specifically, the comfort enhancing ingredient can be at least one selected from gamma-polyglutamic acid (γ-PGA) and its salts and gamma-polyglutamate, and the molecular weight thereof can be between 3,000 Daltons and 1,500,000 Daltons. The content of the comfort enhancing ingredient is between 0.01 wt % and 3 wt % based on 100 wt % of the contact lens solution, e.g., 0.05 wt %, 0.1 wt %, 0.2 wt %, 0.3 wt %, 0.4 wt %, 0.5 wt %, 0.6 wt %, 0.7 wt %, 0.8 wt %, 0.9 wt %, 1.0 wt %, 1.5 wt %, 2.0 wt % or 2.5 wt %.

It should be noted that, when the contact lens solution of the present disclosure is in sufficient contact with a contact lens, a thin film of the comfort enhancing ingredient can be formed on a surface of the contact lens. The thin film has good oxygen permeability, surface hydrophilicity, wettability and lubricity, such that the wearing comfort of the contact lens can be efficiently increased.

The contact lens solution of the present disclosure can further include a buffering agent, a surfactant, a hydrophilic polymer and other functional additives. The buffering agent can be added to adjust the pH and osmolality of the contact lens solution to allow the contact lens solution to have expected effects, i.e., beneficial effects for the eyes. The pH of the contact lens solution can be from 6 to 8, and the osmolality of the contact lens solution can be from 250 Osmol/Kg to 450 Osmol/Kg.

In the present embodiment, the buffering agent is a borate buffer or a phosphate buffer. The content of the buffering agent is greater than 0 wt % and less than 5 wt %, based on 100 wt % of the contact lens solution, e.g., 0.5 wt %, 1 wt %, 1.5 wt %, 2 wt %, 2.5 wt %, 3 wt %, 3.5 wt % or 4 wt %. The borate buffer may include boric acid, sodium chloride and borate(s) (e.g., sodium tetraborate), but it is not limited thereto. The phosphate buffer may include sodium chloride and phosphate(s) (e.g., sodium dihydrogen phosphate, disodium hydrogen phosphate, potassium dihydrogen phosphate and dipotassium hydrogen phosphate), but it is not limited thereto.

The surfactant can be added to enhance the performance of the comfort enhancing ingredient. The surfactant can be at least one selected from polysorbate 80 (also known as Tween 80), an alkyl sulfosuccinate (e.g., SBFA 30), polyethylene glycol, polyoxypropylene glycol, and sodium dodecyl sulfate (also called “sodium lauryl sulfate”), but it is not limited thereto. The content of the surfactant is between 0.01 wt % and 2 wt %, preferably between 0.01 wt % and 1 wt %, based on 100 wt % of the contact lens solution, e.g., 0.05 wt %, 0.1 wt %, 0.2 wt %, 0.3 wt %, 0.4 wt %, 0.5 wt %, 0.6 wt %, 0.7 wt %, 0.8 wt % or 0.9 wt %.

The hydrophilic polymer can be added to increase eye moisture. The hydrophilic polymer can be at least one selected from polyvinyl alcohol (PVA), cellulose and its derivatives, polyethylene glycol (PEG), palmitic acid, polyvinylpyrrolidone (PVP) and its copolymers, 2-methacryloyloxyethyl phosphorylcholine (MPC) and its copolymers, and hyaluronic acid, but it is not limited thereto. The content of the hydrophilic polymer is between 0.01 wt % and 5 wt %, preferably between 0.01 wt % and 3 wt %, based on 100 wt % of the contact lens solution, e.g., 0.5 wt %, 1 wt %, 1.5 wt %, 2 wt % or 2.5 wt %. Specific examples of the cellulose derivative include hydroxypropyl methylcellulose (HPMC), carboxymethyl cellulose (CMC), methyl cellulose (MC), hydroxyethyl cellulose (HEC) and cationic cellulose derivatives.

The functional additive can be at least one selected from an antibacterial agent, a vitamin, a flavonoid substance and a polyphenol substance, but it is not limited thereto. The content of the functional additive may be between 0.01 wt % and 5 wt % based on 100 wt % of the contact lens solution. Specific examples of the antimicrobial agent include polyhexamethylene biguanide (PHMB) and its water soluble salts and polyaminopropyl biguanide (PAPB) and its water soluble salts. Specific examples of the vitamin include vitamin B6 (pyridoxine hydrochloride), vitamin B12 (cyanocobalamin) and vitamin E (synthetic dl-alpha-tocopherol). However, the above examples are not intended to limit the present disclosure.

Table 1 shows representative examples of the contact lens solutions of the present disclosure. However, the present disclosure is not limited to these examples.

TABLE 1

| Ingredients | Example 1 | Example 2 | Example 3 | Example 4 | Example 5 | Example 6 | Example 7 Example 8 | |
|---|---|---|---|---|---|---|---|---|
| Boric acid | 0.50% | 0.50% | 0.50% | 0.50% | 0.50% | 0.50% | 0.50% | 0.50% |
| Sodium | 0.80% | 0.80% | 0.80% | 0.80% | 0.80% | 0.80% | 0.80% | 0.80% |
| chloride | | | | | | | | |
| Sodium | 0.03% | 0.03% | 0.03% | 0.03% | 0.03% | 0.03% | 0.03% | 0.03% |
| tetraborate | | | | | | | | |
| Polysorbate | 0.50% | 0.50% | 0.50% | 0.50% | 0.50% | 0.50% | 0.50% | 0.50% |
| Polyethylene | 0.50% | 0.50% | 0.50% | 0.50% | 0.50% | 0.50% | 0.50% | 0.50% |
| glycol | | | | | | | | |
| Vitamin | | | | | | | 0.30% | 0.30% |
| Hyaluronic | 0.05% | 0.20% | | | | | | |
| acid | | | | | | | | |
| poly MPC | | | 3.00% | | | | | |
| γ-PGA | | | | 0.05% | 0.10% | 0.20% | | 0.2% |

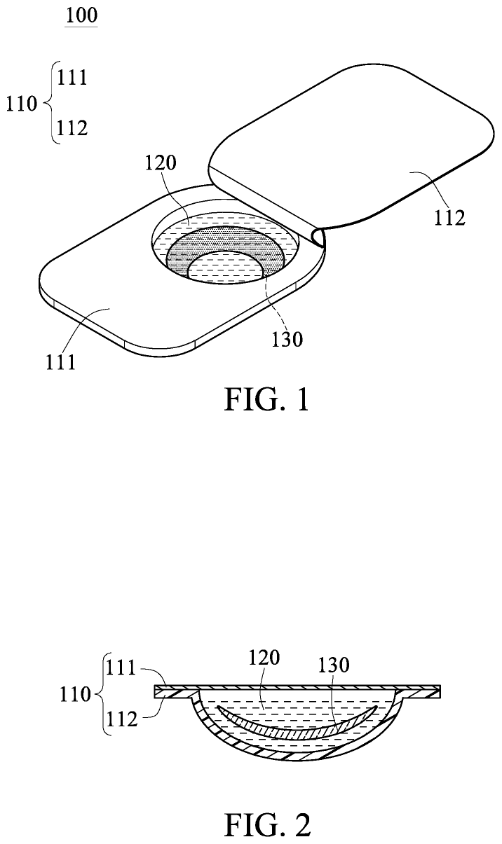

Reference is made to FIG. 1 and FIG. 2. In one preferred embodiment, the contact lens product 100 of the present disclosure is a contact lens package 100 that includes a package structure 110, a contact lens solution 120 and a contact lens 130. The contact lens solution 120 and the contact lens 130 are sealed together in the package structure 110 and are sterilized (e.g., sterilized at high temperature or high pressure), in which the contact lens 130 is immersed in the contact lens solution 120.

More specifically, the package structure 110 includes a container 111 and a cover sheet 112. The container 111 is used to accommodate the contact lens solution 120 and the contact lens 130, and the cover sheet 112 is peelably bonded to the container 111 to seal an opening of the container 111. In the present embodiment, the container 111 may be made of a plastic, and provides a reasonable degree of protection to the contact lens 130. The cover sheet 112 may be made of a metal or a plastic. The contact lens 130 may be made of a hydrogel or a silicone hydrogel, and may contain one or more functional materials (e.g., a blue light absorbing material or a UV absorbing material) if necessary. However, the above examples are not intended to limit the present disclosure.

It should be noted that, when the contact lens 130 is immersed in the contact lens solution 120, the comfort enhancing ingredient of the contact lens solution 120 would enter the contact lens 130 or adhere onto the contact lens 130 to produce moisture retention and lubrication effects, thereby increasing the wearing comfort. The contact lens solution 120 of the present disclosure is particularly suitable for people who wear contact lenses for a long period of time.

### Evaluation Areas

Preparation of Contact Lens Products:

Contact lens solutions serving as storage solutions were prepared according to the compositions as shown in Table 1. Hydrogel contact lenses produced by the Pegavision Corporation were respectively immersed in the contact lens solutions of Examples 1-8. After sealing and high temperature sterilizing (125° C., 30 minutes) treatments, the contact lens products were obtained. The surface properties of the hydrogel contact lenses were scored from 1 to 10 by ten individuals in a blind test, in which a high score represents better lubricity than a low score, and the results were shown in Table 2 below.

TABLE 2

| Examples | 1 | 2 | 3 | 4 | 5 | 6 | 7 | 8 |
|---|---|---|---|---|---|---|---|---|
| Score of | 4.5 | 4.8 | 6.6 | 5.8 | 6.4 | 6.8 | 4.6 | 6.5 |
| lubricity | | | | | | | | |

Water Film Breakup Time (WBUT) Test:

Contact lens solutions serving as storage solutions were prepared according to the compositions as shown in Table 1. Hydrogel contact lenses produced by the Pegavision Corporation were respectively immersed in the contact lens solutions of Examples 1-8. These contact lenses were taken out from the solutions and their water film breakup times were recorded, as shown in Table 3. It is observed from Table 3 that, the contact lens solutions each having an effective amount of gamma-polyglutamic acid have extended water film breakup times.

TABLE 3

| Examples | 1 | 2 | 3 | 4 | 5 | 6 | 7 | 8 |
|---|---|---|---|---|---|---|---|---|
| WBUT | 39-61 | 44-61 | 50-67 | 47-61 | 61-71 | 63-70 | 40-56 | 58-68 |
| (seconds) | | | | | | | | |

Clinical Test:

Contact lens solutions serving as storage solutions were prepared according to the compositions as shown in Table 1. Hydrogel contact lenses produced by the Pegavision Corporation were respectively immersed in the contact lens solutions of Examples 2-8. These contact lenses were taken out from the solutions and then put on users' eyes. Eleven users were selected to evaluate certain items including foreign matter and dryness sensations and the results were shown in Table 4 below. In Table 4, a high discomfort score represents more apparent foreign matter and dryness sensations, and a large number of non-invasive breakup time (NIBUT) represents a moister state of the user's eyes.

TABLE 4

| Evaluate | | Examples | | | | | | |
|---|---|---|---|---|---|---|---|---|
| items | Wear- time | 2 | 3 | 4 | 5 | 6 | 7 | 8 |
| Discomfort | Immediately after | 4 | 1 | 1 | 3 | 1 | 3 | 2 |
| score | putting on | | | | | | | |
| | contact lenses | | | | | | | |
| | After wearing for | 12 | 5 | 9 | 14 | 4 | 11 | 6 |
| | 6 hours | | | | | | | |
| NIBUT | Immediately after | 13.89 | 14.34 | 14.00 | 14.25 | 14.99 | 13.55 | 14.55 |
| (seconds) | putting on | | | | | | | |
| | contact lenses | | | | | | | |
| | After wearing for | 13.30 | 13.13 | 13.15 | 13.41 | 14.03 | 13.10 | 13.94 |
| | 6 hours | | | | | | | |

One of the advantages of the present disclosure is that the contact lens solution for enhancing moist sensation and the contact lens product, in which the comfort enhancing ingredient is present in an amount of 0.01-3 wt % and has one or more glutamic acid monomers, can increase the wearing comfort of contact lenses as well as maintain healthy corneas.

Furthermore, a thin film of the comfort enhancing ingredient can be formed on a surface of the contact lens, which has good oxygen permeability, surface hydrophilicity, wettability and lubricity. Therefore, the contact lens solution of the present disclosure is particularly suitable for people who wear contact lenses for a long period of time.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A contact lens solution for enhancing moist sensation, comprising a comfort enhancing ingredient, the content of which is between 0.01 wt % and 3 wt % based on 100 wt % of the contact lens solution, the comfort enhancing ingredient having one or more glutamic acid monomers and having a number average molecular weight between 3,000 Daltons and 1,500,000 Daltons, wherein the contact lens solution has a viscosity between 1 cPs and 3 cPs.

2. The contact lens solution according to claim 1, wherein the comfort enhancing ingredient is at least one selected from gamma-polyglutamic acid (γ-PGA) and its salts and gamma-polyglutamate.

3. The contact lens solution according to claim 1, further comprising a surfactant, the content of which is between 0.01 wt % and 2 wt % based on 100 wt % of the contact lens solution, wherein the surfactant is at least one selected from polysorbate, an alkyl sulfosuccinate, polyethylene glycol, polyoxypropylene glycol and sodium dodecyl sulfate.

4. The contact lens solution according to claim 1, further comprising a buffering agent, the content of which is between 0.01 wt % and 5 wt % based on 100 wt % of the contact lens solution, wherein the buffering agent is a borate buffer or a phosphate buffer.

5. The contact lens solution according to claim 4, wherein the borate buffer includes at least one of boric acid, sodium chloride and sodium borate.

6. The contact lens solution according to claim 4, wherein the borate buffer includes at least one of sodium chloride, sodium dihydrogen phosphate, disodium hydrogen phosphate, potassium dihydrogen phosphate and dipotassium hydrogen phosphate.

7. The contact lens solution according to claim 1, further comprising a hydrophilic polymer, the content of which is between 0.01 wt % and 5 wt % based on 100 wt % of the contact lens solution, wherein the hydrophilic polymer is at least one selected from polyvinyl alcohol, cellulose and its derivatives, polyethylene glycol, palmitic acid, polyvinylpyrrolidone and its copolymers, 2-methacryloyloxyethyl phosphorylcholine and its copolymers, and hyaluronic acid.

8. The contact lens solution according to claim 1, further comprising a functional additive, the content of which is between 0.01 wt % and 5 wt % based on 100 wt % of the contact lens solution, wherein the functional additive is an antimicrobial agent, a vitamin or the combination thereof.

9. The contact lens solution according to claim 1, having a pH of 6 to 8 and an osmolality of 250 Osmol/Kg to 450 Osmol/Kg.

10. A contact lens product comprising a package provided with the contact lens solution as claimed in claim 1 and a contact lens, the contact lens being immersed in the contact lens solution and sealed in the package.
